(19) United States
(12) Patent Application Publication
(10) Pub. No.: US 2026/0053995 A1
(43) Pub. Date: Feb. 26, 2026

(54) PERITONEAL DIALYSIS SYSTEM AND METHOD FOR MANAGING PATIENT DRAIN PAIN

(71) Applicants: BAXTER INTERNATIONAL INC., Deerfield, IL (US); BAXTER HEALTHCARE SA, Glattpark (Opfikon) (CH)

(72) Inventors: Lalu NMI KRISHNAN, Bangalore, Karnataka (IN); Kamath SADASHIVA, Bangalore, Karnataka (IN)

(21) Appl. No.: 19/106,030

(22) PCT Filed: Aug. 2, 2023
(86) PCT No.: PCT/US2023/029280

§ 371 Date: Feb. 24, 2025

(30) Foreign Application Priority Data
Aug. 24, 2022 (IN) 202241048243

(51) Int. Cl. A61M 1/28

(52) U.S. Cl. CPC A61M 1/282; A61M 1/281; A61M 2202/0014; A61M 2202/04; A61M 2205/3344; A61M 2205/3553; A61M 2205/3592; A61M 2205/502; A61M 2205/6009; A61M 2205/6072

(57) ABSTRACT

A peritoneal dialysis (“PD”) system includes a PD fluid pump; a patient line for receiving used PD fluid pumped by the PD fluid pump during a patient drain; a control unit configured to cause the PD fluid pump to pump, according to a first commanded pressure, the used PD fluid through the patient line during the patient drain; and a personal digital assistant (“PDA”) in wireless communication with the control-unit, the PDA configured to enable a patient while undergoing the patient drain to send a command to the control unit, the command instructing the control unit to cause the PD fluid to pump according to a second commanded pressure during the patient drain.

### BACKGROUND

The present disclosure relates generally to medical fluid treatments and in particular to dialysis fluid treatments.

Due to various causes, a person's renal system can fail. Renal failure produces several physiological derangements. It is no longer possible to balance water and minerals or to excrete daily metabolic load. Toxic end products of metabolism, such as, urea, creatinine, uric acid and others, may accumulate in a patient's blood and tissue.

Reduced kidney function and, above all, kidney failure is treated with dialysis. Dialysis removes waste, toxins and excess water from the body that normal functioning kidneys would otherwise remove. Dialysis treatment for replacement of kidney functions is critical to many people because the treatment is lifesaving.

One type of kidney failure therapy is Hemodialysis (“HD”), which in general uses diffusion to remove waste products from a patient's blood. A diffusive gradient occurs across the semi-permeable dialyzer between the blood and an electrolyte solution called dialysate or dialysis fluid to cause diffusion.

Hemofiltration (“HF”) is an alternative renal replacement therapy that relies on a convective transport of toxins from the patient's blood. HF is accomplished by adding substitution or replacement fluid to the extracorporeal circuit during treatment. The substitution fluid and the fluid accumulated by the patient in between treatments is ultrafiltered over the course of the HF treatment, providing a convective transport mechanism that is particularly beneficial in removing middle and large molecules.

Hemodiafiltration (“HDF”) is a treatment modality that combines convective and diffusive clearances. HDF uses dialysis fluid flowing through a dialyzer, similar to standard hemodialysis, to provide diffusive clearance. In addition, substitution solution is provided directly to the extracorporeal circuit, providing convective clearance.

Most HD, HF, and HDF treatments occur in centers. A trend towards home hemodialysis (“HHD”) exists today in part because HHD can be performed daily, offering therapeutic benefits over in-center hemodialysis treatments, which occur typically bi- or tri-weekly. Studies have shown that more frequent treatments remove more toxins and waste products and render less interdialytic fluid overload than a patient receiving less frequent but perhaps longer treatments. A patient receiving more frequent treatments does not experience as much of a down cycle (swings in fluids and toxins) as does an in-center patient, who has built-up two or three days' worth of toxins prior to a treatment. In certain areas, the closest dialysis center can be many miles from the patient's home, causing door-to-door treatment time to consume a large portion of the day. Treatments in centers close to the patient's home may also consume a large portion of the patient's day. HHD can take place overnight or during the day while the patient relaxes, works or is otherwise productive.

Another type of kidney failure therapy is peritoneal dialysis (“PD”), which infuses a dialysis solution, also called dialysis fluid, into a patient's peritoneal chamber via a catheter. The dialysis fluid is in contact with the peritoneal membrane in the patient's peritoneal chamber. Waste, toxins and excess water pass from the patient's bloodstream, through the capillaries in the peritoneal membrane, and into the dialysis fluid due to diffusion and osmosis, i.e., an osmotic gradient occurs across the membrane. An osmotic agent in the PD dialysis fluid provides the osmotic gradient. Used or spent dialysis fluid is drained from the patient, removing waste, toxins and excess water from the patient. This cycle is repeated, e.g., multiple times.

There are various types of peritoneal dialysis therapies, including continuous ambulatory peritoneal dialysis (“CAPD”), automated peritoneal dialysis (“APD”), tidal flow dialysis and continuous flow peritoneal dialysis (“CFPD”). CAPD is a manual dialysis treatment. Here, the patient manually connects an implanted catheter to a drain to allow used or spent dialysis fluid to drain from the peritoneal chamber. The patient then switches fluid communication so that the patient catheter communicates with a bag of fresh dialysis fluid to infuse the fresh dialysis fluid through the catheter and into the patient. The patient disconnects the catheter from the fresh dialysis fluid bag and allows the dialysis fluid to dwell within the peritoneal chamber, wherein the transfer of waste, toxins and excess water takes place. After a dwell period, the patient repeats the manual dialysis procedure, for example, four times per day. Manual peritoneal dialysis requires a significant amount of time and effort from the patient, leaving ample room for improvement.

Automated peritoneal dialysis (“APD”) is similar to CAPD in that the dialysis treatment includes drain, fill and dwell cycles. APD machines, however, perform the cycles automatically, typically while the patient sleeps. APD machines free patients from having to manually perform the treatment cycles and from having to transport supplies during the day. APD machines connect fluidly to an implanted catheter, to a source or bag of fresh dialysis fluid and to a fluid drain. APD machines pump fresh dialysis fluid from a PD fluid source, through the catheter and into the patient's peritoneal chamber. APD machines also allow for the dialysis fluid to dwell within the chamber and for the transfer of waste, toxins and excess water to take place. The source may include multiple liters of dialysis fluid including several solution bags.

APD machines pump used or spent dialysate from the patient's peritoneal cavity, though the catheter, to drain. As with the manual process, several drain, fill and dwell cycles occur during dialysis. A “last fill” may occur at the end of the APD treatment. The last fill fluid may remain in the peritoneal chamber of the patient until the start of the next treatment, or may be manually emptied at some point during the day.

APD patients may experience what is known as drain pain. The negative pressure applied to the patient's peritoneal cavity during drain may cause pain, especially for new patients where the peritoneal cavity may be extra sensitive. The pain level may be to the level that the patient no longer wishes to continue an APD treatment. Certain APD machines are configured to supply negative pumping pressure using pneumatic or air pressure applied to an air side of one or more flexible membrane or diaphragm. The negative pressure sucks the flexible membrane or diaphragm inwardly, causing used PD fluid or effluent to be pulled from the patient along a fluid side of the flexible membrane or diaphragm. The negative pneumatic pressure applied is constant, which means that the applied pressure is the same regardless of the conditions present at the patient, which are not constant.

The conditions present at the patient change. For example, the patients' sleeping position changes assuming a nighttime treatment in which the patient is sleeping. Also, the volume of effluent residing within the patient's peritoneal cavity changes over the course of a patient drain. Each of these changing conditions affects the flowrate of used PD fluid or effluent flowing along the patient line back to the APD machine. The flowrate affects the amount of pressure drop along the patient line, where the higher the flowrate the higher the pressure drop.

In low flow patient conditions, e.g., where the patient's sleeping position has somehow limited drain flowrate, or the patient's peritoneal cavity is almost empty, creating a lack of effluent supply situation, the corresponding pressure drop will be low. A constant negative pressure applied for both high and low drain flowrates will create an environment for patient drain pain during low drain flowrates.

A need exists accordingly for an improved APD system that mitigates or eliminates patient drain pain.

### SUMMARY

The present disclosure sets forth an automated peritoneal dialysis (“PD”) system that mitigates or eliminates drain pain. The PD system includes a PD machine or cycler. PD treatments using the PD machine occur in cycles, each cycle having a fill phase, a dwell phase and a drain phase. During the fill phase, fresh PD fluid is delivered to the patient under positive pressure. During the dwell phase, the fluid just filled dwells or sits within and treats the patient. During the drain phase, used PD fluid is removed from the patient under negative pressure.

The PD machine is in one embodiment capable of delivering fresh, heated PD fluid to the patient at, for example, 14 kPa (2.0 psig) or higher. The PD machine is capable of removing used PD fluid or effluent from the patient at, for example, −9 kPa (−1.3 psig) or an even greater negative pressure. The resulting flowrate to or from the patient may be dependent on a number of factors, including where the patient is located elevationally compared to the APD machine's pumping portion, the patient's sleeping position (assuming a nighttime treatment), and for a patient drain, the amount of effluent remaining in the patient's peritoneal cavity.

If the PD machine pulls too hard during the drain phase, the patient may experience pain at the patient's sensitive peritoneal cavity. Drain pain occurs often at the end of the drain when there is less used PD fluid to be pulled by the machine's suction. Drain pain may also occur at or due to a misplaced PD catheter, which resides within the patient. The level of drain pain is only known to the patient experiencing it. The pain may be significant enough that the patient eventually looks for an alternative kidney failure treatment.

To mitigate drain pain, a mobile application accessed via the patient's smartphone or a notebook/wireless keypad according to the present disclosure is provided. The smartphone or notebook/wireless keypad (which may be called a personal digital assistant (“PDA”)) is synched to the PD machine, e.g., via WIFI, Bluetooth or other wireless technology. The wireless communication allows the patient during drain to control the amount of negative pressure or suction provided by the machine, e.g., to lower the negative pressure or suction if the patient is experiencing drain pain. To do so, the PD machine's control unit receives the wireless communication and causes the PD fluid pump to modify its negative output pressure accordingly.

The smartphone or notebook/wireless keypad having the mobile application of the present disclosure in one embodiment provides and displays “Up” and “Down” buttons or selectors that enable the patient to increase and decrease the PD machine's negative pressure or suction level between a minimum allowable negative pressure and a maximum allowable negative pressure. The display screen of the smartphone or notebook/wireless keypad displays a negative pressure level number, e.g., 1 to 10, which is a relative scale, wherein 1 indicates the minimum allowable negative pressure, 10 indicates the maximum allowable negative pressure, and 2 to 9 range from the minimum to the maximum pressures.

The “Up” and “Down” buttons or selectors increment the count one-by-one in the selected direction. If the patient for example begins to experience a lot of drain pain while the PD machine is pumping at a high negative pressure level, the patient may press a “Min” or minimum button, which causes the pump to modify its output such that the negative pressure is lowered from whatever level it is at to the minimum negative pressure level of 1. If the patient instead feels good and wants to speed-up the drain phase by raising the negative pressure level, the patient may press the “Max” or maximum button, which causes the pump to modify its output such the negative pressure is raised from whatever level it is at to the maximum negative pressure level of 10. Negative pressure levels 1 to 10 are in one embodiment each within established safe patient pressure limits so that in no situation is it possible for the PD machine to supply too much negative pressure, potentially overpressurizing the patient, or too little negative pressure, leading to an ineffective patient drain.

The smartphone or notebook/wireless keypad also provides a Send button. When a negative pressure value that the patient wishes to send to the PD machine has been selected, the patient selects or presses the Send button, which causes the smartphone or notebook/wireless keypad to wirelessly deliver the new commanded negative pressure value to the control unit of the PD machine. The patient may therefore increment negative pressure values at the smartphone or notebook/wireless keypad without those values being sent to the PD machine. The pressing of the Send button causes the negative pressure value currently displayed at the smartphone or notebook/wireless keypad to be delivered to the PD machine, which changes the pumping of used PD fluid from the patient accordingly. The smartphone or notebook/wireless keypad may optionally display a popup confirm button after the patient presses the Send button to confirm that the patient wants a new negative pressure value to be delivered to the PD machine.

The PD machine may employ pneumatic PD fluid pumping or electromechanical PD fluid pumping, for example. With pneumatic PD fluid pumping, the negative pressure level may be adjusted by adjusting a variable orifice of a pressure regulator. With pneumatic pumping, the negative pressure of the PD fluid may be known by measuring the pneumatic driving pressure, which is the same as the PD fluid pressure. If the PD fluid pumping is instead electromechanical, e.g., via a piston pump, the negative pressure level may be adjusted by adjusting the speed of a motor, such as a stepper motor driving the electromechanical pump. With electromechanical pumping, the negative pressure of the PD fluid may be known by placing a pressure sensor along (or in fluid communication with) the negative pressure PD fluid line leading to the electromechanical pump.

In light of the disclosure set forth herein, and without limiting the disclosure in any way, in a first aspect of the present disclosure, which may be combined with any other aspect, or portion thereof, a peritoneal dialysis (“PD”) system includes a PD fluid pump; a patient line for receiving used PD fluid pumped by the PD fluid pump during a patient drain; a control unit configured to cause the PD fluid pump to pump, according to a first commanded pressure, the used PD fluid through the patient line during the patient drain; and a personal digital assistant (“PDA”) in wireless communication with the control unit, the PDA configured to enable a patient while undergoing the patient drain to send a command to the control unit, the command instructing the control unit to cause the PD fluid pump to pump the used PD fluid according to a second commanded pressure during the patient drain.

In a second aspect of the present disclosure, which may be combined with any other aspect, or portion thereof, the second commanded pressure is less than the first commanded pressure, the second commanded pressure aiding in the reduction of pain experienced by the patient during the patient drain.

In a third aspect of the present disclosure, which may be combined with any other aspect, or portion thereof, the second commanded pressure is greater than the first commanded pressure, the second commanded pressure aiding in reducing a duration of the patient drain.

In a fourth aspect of the present disclosure, which may be combined with any other aspect, or portion thereof, the PDA provides a set of relative pressure values from which the patient may choose for sending the command, and wherein the control unit is configured to convert a selected relative pressure value to the second commanded pressure.

In a fifth aspect of the present disclosure, which may be combined with any other aspect, or portion thereof, the PDA provides a set of relative pressure values from which the patient may choose in sending the command, and wherein the control unit is configured to convert a selected relative pressure value to a pump actuation value that corresponds to the second commanded pressure.

In a sixth aspect of the present disclosure, which may be combined with any other aspect, or portion thereof, the PDA provides a set of relative pressure values from which the patient may choose for sending the command, and wherein the PDA is configured to convert a selected relative pressure value to the second commanded pressure.

In a seventh aspect of the present disclosure, which may be combined with any other aspect, or portion thereof, the PDA provides a set of relative pressure values from which the patient may choose for sending the command, and wherein the PDA is configured to convert a selected relative pressure value to a pump actuation value that corresponds to the second commanded pressure.

In an eighth aspect of the present disclosure, which may be combined with any other aspect, or portion thereof, the PDA provides a set of relative pressure values from which the patient may choose for sending the command, and wherein the PDA provides at least one of an incremental up selector or an incremental down selector for incrementing through the set of relative pressure values.

In a ninth aspect of the present disclosure, which may be combined with any other aspect, or portion thereof, the PDA further provides a display for displaying an incremented relative pressure value.

In a tenth aspect of the present disclosure, which may be combined with any other aspect, or portion thereof, the PDA further provides a selector for causing an incremented relative pressure value desired by the patient, or a conversion of the incremented relative pressure value, to be delivered to the control unit.

In an eleventh aspect of the present disclosure, which may be combined with any other aspect, or portion thereof, the PDA further provides a selector for moving through the set of relative pressure values to a minimum relative pressure value.

In a twelfth aspect of the present disclosure, which may be combined with any other aspect, or portion thereof, the PDA further provides a selector for moving through the set of relative pressure values to a maximum relative pressure value.

In a thirteenth aspect of the present disclosure, which may be combined with any other aspect, or portion thereof, the PD fluid pump is pneumatically operated or electromechanically operated.

In a fourteenth aspect of the present disclosure, which may be combined with any other aspect, or portion thereof, the PDA is configured to be registered with the control unit such that after registration, the PDA and the control unit communicate wirelessly automatically upon being powered.

In a fifteenth aspect of the present disclosure, which may be combined with any other aspect, or portion thereof, the PDA is configured to be registered with the control unit via a serial number or QR code from the PDA being entered into the control unit.

In a sixteenth aspect of the present disclosure, which may be combined with any other aspect, or portion thereof, the PDA runs a mobile application configured to enable the patient while undergoing the patient drain to send the command to the control unit.

In a seventeenth aspect of the present disclosure, which may be combined with any other aspect, or portion thereof, the command is a first command, wherein the control unit is further configured to communicate with a patient clinic over a network, and wherein the PDA further provides a selector configured such that when selected a second command is generated requesting that the control unit contact the patient clinic.

In an eighteenth aspect of the present disclosure, which may be combined with any other aspect, or portion thereof, the PD fluid pump and the control unit are part of a PD machine, and wherein the PDA is remote from the PD machine.

In a nineteenth aspect of the present disclosure, which may be combined with any other aspect, or portion thereof, a peritoneal dialysis (“PD”) system includes a PD fluid pump; a patient line for receiving PD fluid pumped by the PD fluid pump during a PD treatment procedure; a control unit configured to cause the PD fluid pump to pump, according to a first commanded pressure, the PD fluid through the patient line during the PD treatment procedure; and a personal digital assistant (“PDA”) in wireless communication with the control unit, the PDA configured to enable a patient while undergoing the PD treatment procedure to send a command to the control unit, the command instructing the control unit to cause the PD fluid pump to pump PD fluid according to a second commanded pressure during the PD treatment procedure.

In a twentieth aspect of the present disclosure, which may be combined with any other aspect, or portion thereof, the PD fluid is used PD fluid and the PD treatment procedure is a patient drain.

In a twenty-first aspect of the present disclosure, which may be combined with any other aspect, or portion thereof, a peritoneal dialysis (“PD”) machine includes a PD fluid pump; a patient line for receiving used PD fluid pumped by the PD fluid pump during a patient drain; a control unit configured to cause the PD fluid pump to pump, according to a first commanded pressure, the used PD fluid through the patient line during the patient drain; and a user interface in communication with the control unit, the user interface configured to enable a patient while undergoing the patient drain to send a command to the control unit, the user interface command instructing the control unit to cause the PD fluid pump to pump the used PD fluid according to a second commanded pressure during the patient drain.

In a twenty-second aspect of the present disclosure, which may be combined with any other aspect, or portion thereof, a PD system includes a personal digital assistant (“PDA”) in wireless communication with the control unit, the PDA also configured to enable the patient while undergoing the patient drain to send a command to the control unit, the PDA command instructing the control unit to cause the PD fluid pump to pump the used PD fluid according to a second commanded pressure during the patient drain.

In a twenty-third aspect of the present disclosure, which may be combined with any other aspect, or portion thereof, any of the features, functionality and alternatives described in connection with any one or more of FIGS. 1 to 5 may be combined with any of the features, functionality and alternatives described in connection with any other of FIGS. 1 to 5.

In light of the above aspects and present disclosure set forth herein, it is an advantage of the present disclosure to provide a system and method for an automated peritoneal dialysis (“PD”) machine or cycler that allows for the minimization of patient drain pain.

It is another advantage of the present disclosure to provide a system and method for a PD machine or cycler that provides patient control to drain pressure management.

It is a further advantage of the present disclosure to provide a system and method for a PD machine or cycler that allows for the minimization of patient drain pain without requiring additional PD machine equipment.

Moreover, it is an advantage of the present disclosure to provide a system and method for drain pressure control, which is easy for the patient to use.

Additional features and advantages are described in, and will be apparent from, the following Detailed Description and the Figures. The features and advantages described herein are not all-inclusive and, in particular, many additional features and advantages will be apparent to one of ordinary skill in the art in view of the figures and description. Also, any particular embodiment does not have to have all of the advantages listed herein and it is expressly contemplated to claim individual advantageous embodiments separately. Moreover, it should be noted that the language used in the specification has been selected principally for readability and instructional purposes, and not to limit the scope of the inventive subject matter.

### BRIEF DESCRIPTION OF THE FIGURES

FIG. 1 is a sectioned schematic view of one embodiment for a peritoneal dialysis (“PD”) system of the present disclosure, which is configured to allow a patient to have drain pressure control.

FIG. 2 is a schematic view illustrating an alternative electromechanical pump and closed loop pressure control therefore.

FIG. 3 is a schematic illustration of one embodiment of the patient drain pressure controlled PD system of the present disclosure, which includes a PD machine, associated PD fluid containers and patient connection, and a remote personal digital assistant (“PDA”) for implementing drain pressure control.

FIG. 4 is a schematic diagram illustrating remote PDA embodiments in more detail.

FIG. 5 is a process flow diagram illustrating one method for implementing the patient drain pressure control into a PD treatment of the present disclosure.

### DETAILED DESCRIPTION

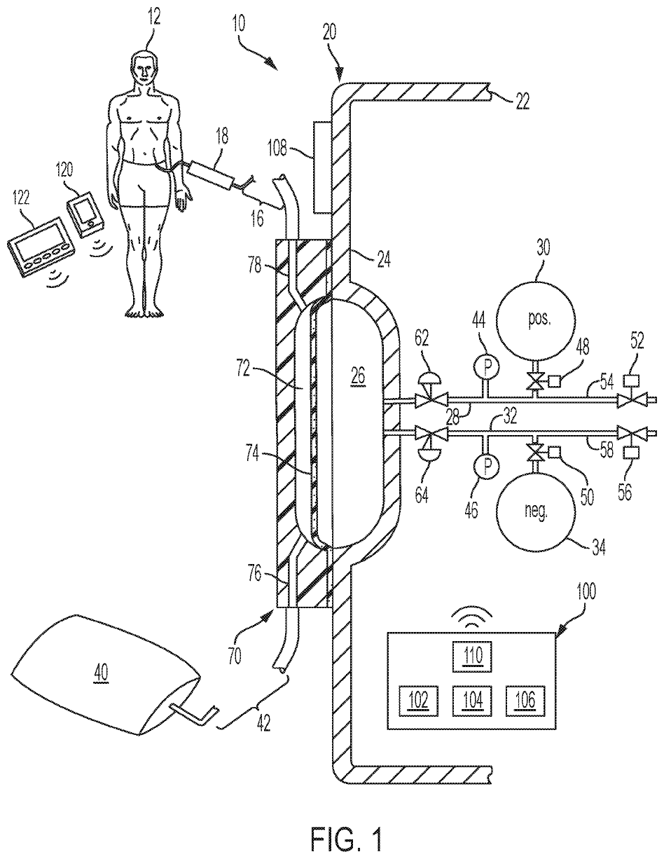

Referring now to the drawings and in particular to FIG. 1, an example system 10 including the patient drain pain reduction methodology of the present disclosure is illustrated. System 10 includes a dialysis machine 20, such as an APD machine, operating a medical fluid handling device 70, such as a dialysis fluid cassette. PD machine 20 in the example of FIG. 1 includes a housing 22 defining a pump interface 24 having a pump actuator or pump actuation area 26 for actuating medical fluid handling device 70. Pump actuation area 26 in the example of FIG. 1 is actuated pneumatically via a positive pneumatic line 28 extending from a positive pneumatic source 30 to perform a pump-out or discharge stroke, e.g., to push (i) fresh, heated dialysis fluid to a peritoneal cavity of patient 12 via a patient line 16 and patient transfer set 18, (ii) fresh dialysis fluid to a heating container (not illustrated) to be heated to body temperature, e.g., 37° C., by a dialysis fluid heater (not illustrated), or (iii) used dialysis fluid to a drain. Patient line 16 may be from three to seven meters long, e.g., approximately 4.5 or 6.7 meters, and have an internal diameter of, for example, three to four millimeters. Pump actuation area 26 in the illustrated embodiment is actuated pneumatically via a negative pneumatic line 32 extending from a negative pneumatic source 34 to perform a pump-in or draw stroke, e.g., to pull (i) fresh dialysis fluid from a PD fluid source 40 through a supply line 42, (ii) fresh, heated dialysis fluid from the heating container (not illustrated), or (iii) used dialysis fluid from the peritoneal cavity of patient 12 via patient line 16 and transfer set 18.

PD machine 20 also provides a pressure sensor 44 for measuring positive pneumatic pressure in positive pneumatic line 28 (corresponding to positive PD fluid pressure) and a pressure sensor 46 for measuring negative pneumatic pressure in negative pneumatic line 32 (corresponding to positive PD fluid pressure). PD machine 20 further includes plural electrically operated pneumatic valves, e.g., valves 48, 50, 52 and 56. Pneumatic valve 48 is positioned in positive pneumatic line 28 to selectively allow positive pressure from source 30 to reach pump actuation area 26. Pneumatic valve 50 is positioned in negative pneumatic line 32 to selectively allow negative pressure from source 34 to reach pump actuation area 26. A vent valve 52 is provided in a vent line 54 in communication with positive pneumatic line 28 to selectively vent positive pressure in line 28 and pump actuation area 26 to atmosphere. A second vent valve 56 is provided in a vent line 58 in communication with negative pneumatic line 32 to selectively vent negative pressure in line 32 and pump actuation area 26 to atmosphere. In an alternative embodiment, a single vent valve and line may be provided to vent both positive and negative pressure from pump actuation area 26 to atmosphere.

FIG. 1 further illustrates that PD machine 20 includes a positive pneumatic pressure regulator 62, e.g., a variable orifice valve, located along positive pneumatic line 28, and a negative pressure regulator 64, e.g., a variable orifice valve, located along negative pneumatic line 32. Positive pneumatic pressure regulator 62 sets the positive pneumatic pressure delivered to pump actuation area 26 to a desired and controlled level, which is also the pressure of fresh or used PD fluid pumped out of dialysis fluid cassette 70. Negative pneumatic pressure regulator 64 sets the negative pneumatic pressure drawn at pump actuation area 26 to a desired and controlled level, which is also the pressure of fresh or used PD fluid pumped into dialysis fluid cassette 70. In an alternative embodiment, PD machine 20 may be configured pneumatically such that a single pressure regulator, e.g., variable orifice valve, operates at different times as a positive pneumatic pressure regulator and a negative pneumatic pressure regulator. In any case, the negative pressure regulator allows the patient to adjust negative PD fluid drain pressure as needed and as described herein.

Pressure sensors 44 and 46, pneumatic valves 48, 50, 52 and 56, and variable orifice valves or regulators 62 and 64 either output to or are under the control of a control unit 100 of PD machine 20. Control unit 100 in the illustrated embodiment includes one or more processor 102, one or more memory 104 and a video controller for displaying images on user interface 108 (which may alternatively or additionally include display screen 132 and the patient drain control input devices of the present disclosure). Control unit 100 may have any one or more of a master controller, safety controller, and/or sub- or delegate controller. Control unit 100 receives pressure readings from pressure sensors 44 and 46 and selectively opens and closes pneumatic solenoid valves 48, 50, 52 and 56 at programmed times or stages. Control unit 100 uses the output of pressure sensors 44 and 46, respectively, in a pressure control routine (e.g., proportional, integral, derivative (“PID”) routine) to control variable orifice valves or regulators 62 and 64 so as to deliver positive and negative pneumatic pressure at a desired or commanded level. It should be appreciated that control unit 100 may operate with additional pressure sensors, temperature sensors, PD fluid valves, and a PD fluid heater, which are not illustrated to simplify FIG. 1.

FIG. 1 further illustrates that control unit 100 includes a two-way transceiver 110 and network stack for sending information to and receiving information from a personal digital assistant (“PDA”) operated by patient 12. The PDA may be any type of smartphone 120 or notebook/wireless keypad 122. The PDA includes a display area for displaying information to patient 12. The PDA also includes input devices (buttons or selectors) for allowing the patient to enter and change data. The entered and changed data is delivered via transceiver 110 to control unit 100, which uses the data to make changes to the operation of PD machine 20 as discussed in detail herein.

In the illustrated embodiment of FIG. 1, medical fluid handling device or disposable cassette 70 is provided with a pump actuation chamber 72 that mates with pump actuation area 26 to form an overall pumping chamber. Medical fluid handling device 70 in the illustrated embodiment includes a flexible membrane, diaphragm or sheet 74, which may be sized to fit pump actuation chamber 72 or be sized to cover a whole side of medical fluid handling device 70 (as illustrated), wherein a portion of the membrane 74 covers pump actuation chamber 72, and wherein such portion may be at least substantially flat or be pre-domed or pre-shaped to fit into one or both pump actuation area 26 and pump actuation chamber 72. Flexible membrane 74 (or separate membranes) may also cover and be used to actuate medical fluid valves (not illustrated), such as (i) a fluid valve positioned and arranged to selectively allow medical fluid to flow from fluid source 40, through supply line 42 and a supply channel 76 of medical fluid handling device 70 to pump actuation chamber 72 and (ii) a fluid valve positioned and arranged to selectively allow medical fluid to flow from pump actuation chamber 72 through a patient channel 78 of medical fluid handling device 70, through patient line 16 and patient transfer set 18 to the peritoneal cavity of patient 12. It should be appreciated that medical fluid handling device 70 may have additional fluid valves, e.g., additional fluid valves for an additional pump actuation chamber 72 (operating in an alternating manner to provide more continuous flow) and additional fluid valves for multiple supply lines 42, a fluid heater line, and/or a drain line, which are not illustrated to simplify FIG. 1.

Control unit 100 causes negative pressure from source 34 to be applied to flexible membrane 74 to pull the sheet against the wall of pump actuation area 26 to correspondingly pull fresh or used PD fluid into pump actuation chamber 72. To do so, control unit causes valves 48, 52 and 56 to be closed and valve 50 to be open. During the filling of pump actuation chamber 72, pressure sensor 46 measures negative pumping pressure, which is used as feedback in a pressure control routine to set the level of negative pneumatic pressure applied at pump actuation area 26 via negative pressure regulator 64.

Control unit 100 causes positive pressure from source 30 to be applied to flexible membrane 74 to push the sheet against the wall of pump actuation chamber 72 to correspondingly push fresh or used PD fluid from pump actuation chamber 72. To do so, control unit 100 causes valves 50, 52 and 56 to be closed and valve 48 to be open. During the discharge of pump actuation chamber 72, pressure sensor 44 measures positive pumping pressure, which is used as feedback in a pressure control routine to set the level of positive pneumatic pressure applied at pump actuation area 26 via positive pressure regulator 62.

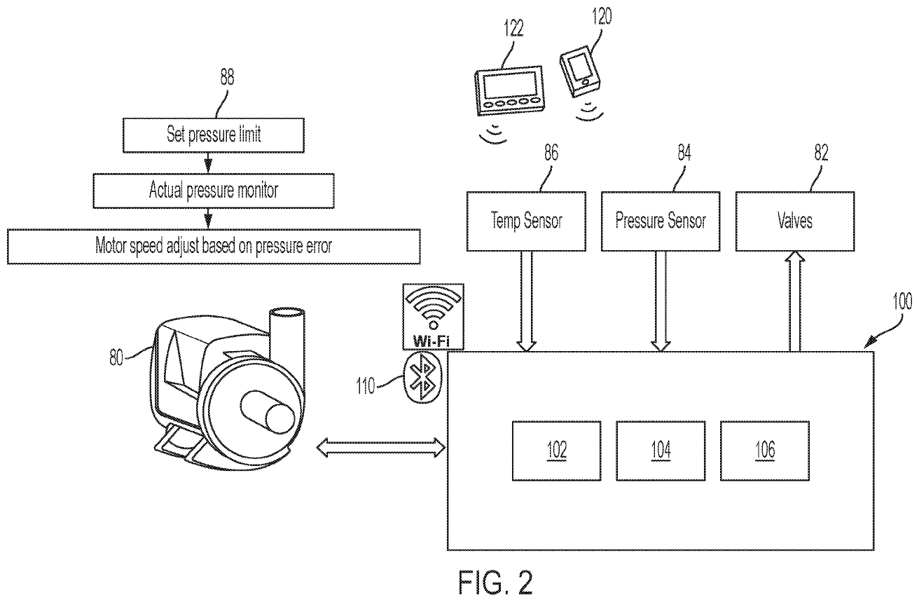

It should be appreciated that it is likely that PD machine 20 provides two pump actuation areas 26 and pump actuation chambers 72, which operate in an alternating manner (one filling while the other discharging), so that the flowrate of fresh or used PD fluid is for the most part continuous. Also, while PD machine 20 if FIG. 1 has been illustrated as a pneumatically operated PD machine, system 10 may alternatively employ different types of PD fluid pumping (and valving), e.g., electromechanical pumping, including piston pump pumping or peristaltic pumping. FIG. 2 illustrates an alternative pumping configuration in which an electromechanical stepper motor pump 80 is employed. In FIG. 2, control unit 100 is employed again, but here to control stepper motor pump 80. Stepper motor pump 80 is in one embodiment a piston pump in which fresh and used PD fluid flows through a body of the pump. Stepper motor pump 80 is in an alternative embodiment a peristaltic pump in which fresh and used PD fluid flows through a tube that is actuated upon by a rotor, which is driven by the stepper motor.

FIG. 2 illustrates that control unit 100 controls valves 82, which are fluid valves, such as electromagnetically operated solenoid pinch valves or motorized pinch valves. Control unit 100 is configured to selectively control valves 82 so as to open flow (i) from a desired fluid source, such as supply container 40, heater container or the patient, (ii) to a desired fluid destination, such as the patient or a drain container/house drain. Control unit 100 receives one or more input from one or more pressure sensor 84, such as first pressure sensor positioned and arranged to sense the pressure of a common PD fluid source line and a second pressure sensor positioned and arranged to sense the pressure of a common PD fluid destination line. Control unit 100 receives one or more input from one or more temperature sensor 86, such as a first temperature sensor positioned and arranged to sense the temperature of PD fluid located within a PD fluid heating container or the PD fluid temperature downstream from an inline PD fluid heater. A second temperature sensor may be provided as a redundant or additional sensor for sensing the temperature of PD fluid located within a PD fluid heating container or be positioned to detect the PD fluid temperature upstream from the inline PD fluid heater.

FIG. 2 further illustrates that one or more memory 104 may store an electromechanical pump algorithm, e.g., a proportional, integral, derivative (“PID”) pump algorithm 88, which is operated by one or more processor 102 of control unit 100. Here, control unit 100 via transceiver 110 may receive a commanded drain pressure from the patient's PDA (smartphone 120 or notebook/wireless keypad 122). The commended drain pressure (which may alternatively come from PD machine 20) is used in PID pump algorithm 88 along with a measured patient drain pressure from a corresponding pressure sensor 84 to determine a pressure error. PID algorithm 88 inputs the pressure error and using different PID gain values outputs a revised speed for stepper motor pump 80, which may be in the form of a current, a pulse-width-modulation (“PWM”) value, or a value associated with the stepper motor controller for obtaining the revise pump speed. Over time, the actual pressure outputted by pressure sensor 84 should meet the commanded pressure.

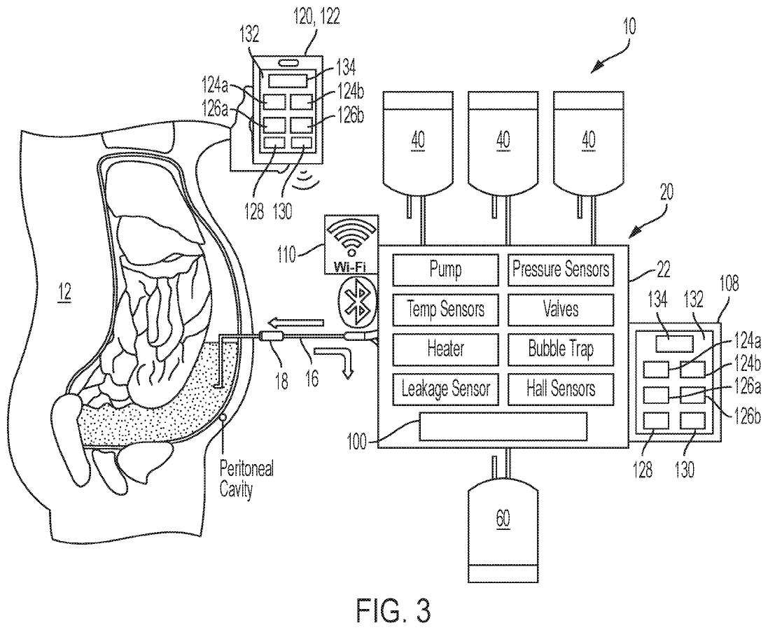

FIG. 3 further schematically illustrates system 10 of the present disclosure. System 10 includes PD machine 20 having a housing that holds any one or more of any of the different types of pumps discussed herein, pneumatic or direct fluid pressure sensors as discussed herein, one or more temperature sensor, pneumatic or electromechanically actuated valves, a batch or inline PD fluid heater, an air or bubble trap to remove air from the PD fluid before being delivered to patient 12, a leakage sensor to detect PD fluid leaks, and/or one or more Hall effect sensor for detecting if a fluid connection is made or if a door of PD machine 20 is properly closed.

In FIG. 3, PD fluid machine receives fresh PD fluid from one or more PD fluid source 40. For a patient fill, control unit 100 causes fresh PD fluid to be delivered from a PD fluid source 40 to the peritoneal cavity of patient 12 via patient line 16, patient transfer set 18 and a peritoneal dialysis catheter 14, which is surgically attached to patient 12 and extends from patient transfer set 18 into the peritoneal cavity of the patient. For a patient drain, control unit 100 causes used PD fluid to be delivered from the peritoneal cavity of patient 12, back through peritoneal dialysis catheter 14, through patient transfer set 18, and through patient line 16 to a drain container 60 or a house drain, such as a nearby toilet or bathtub.

As discussed herein, applying a constant negative pressure during a patient drain may lead to patient drain pain during periods of low flowrate or for reasons associated with the location of peritoneal dialysis catheter 14. To combat drain pain, and to allow the patient to have control over drain pressure generally, present system 10 provides the patient with the wireless PDA (smartphone 120 or notebook/wireless keypad 122) discussed herein, which communicates with control unit 100 via transceiver 110 and WIFI, Bluetooth or other wireless technology. As illustrated in FIG. 3, smartphone 120 and notebook/wireless keypad 122 each include or display a plurality of buttons or selectors. The buttons may be touchscreen buttons formed from a touchscreen overlay placed on a display screen 132. The buttons may alternatively be electromechanical buttons, such as membrane switches. The buttons may include incrementing buttons, such as an Up button 124a and a Down button 124b. The buttons may include limit buttons, such as a Min button 126a and a Max button 126b. The buttons may further include a Help button 128 for when patient drain pain cannot be mitigated and a Send button 130 that the patient presses to send a commanded negative drain pressure update.

FIG. 3 further illustrates that user interface 108 of PD machine 20 may show display screen 132 and all associated user drain control buttons or selectors alternatively, or in addition to, display screen 132 on smartphone 120 or notebook/wireless keypad 122. As illustrated in FIG. 3, display screen 132 at user interface 108 includes Up button 124a, Down button 124b, Min button 126a, Max button 126b, Help button 128, Send button 130 (each selectable by the patient, e.g., via a touchscreen associated with user interface 108) and display area 134 showing a number corresponding to a drain pressure level. In one embodiment, display screen 132 at user interface 108 is displayed along with display screen 132 at smartphone 120 and notebook/wireless keypad 122. A change made by the patient at display screen 132 of any of user interface 108, smartphone 120 or notebook/wireless keypad 122 is reflected at display screen 132 of the other of user interface 108, smartphone 120 or notebook/wireless keypad 122. That is, display screen 132 of user interface 108 is updated to mirror changes made at that of smartphone 120 or notebook/wireless keypad 122 and vice versa.

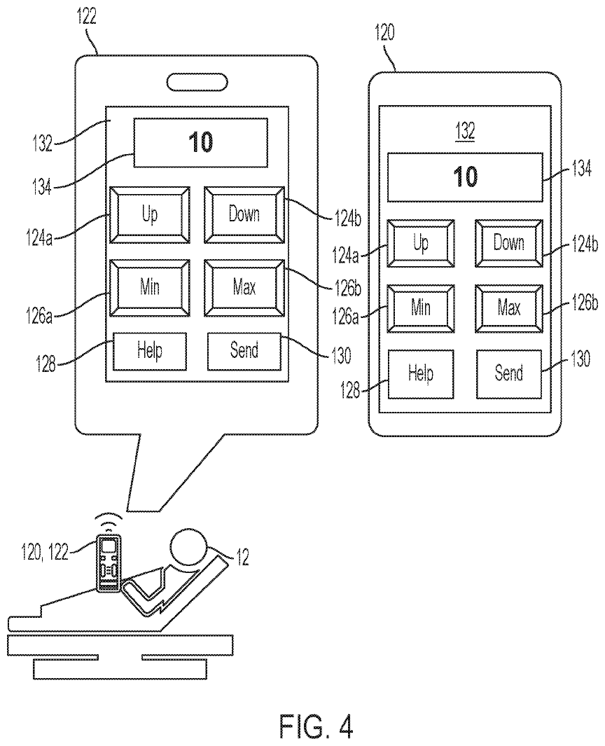

Referring now to FIG. 4, patient 12 is illustrated in a reclined state, e.g., during a PD treatment. The patient is in control of their PDA (smartphone 120 or notebook/wireless keypad 122). Smartphone 120 and notebook/wireless keypad 122 are exploded so that the incrementing buttons or selectors, including Up button 124a and Down button 124b, the limit buttons, such as Min button 126a and Max button 126b, Help button 128 and Send button 130 are displayed in more detail. Display screen 132 is illustrated having a display area 134, which shows a number that corresponds a drain pressure level. The illustrated example for display area 134 shows the numbers one to ten. In other embodiments a smaller scale, such as one to five, or a larger scale, such as one to one-hundred, is provided.

Regarding the wireless keypad 122 version of the patient's PDA, wireless keypad 122 may be a small keypad with few buttons and be powered from one or more coin cell battery. Wireless keypad 122 is in one embodiment provided with a Bluetooth transceiver and a network stack. Wireless keypad 122 communicates with control unit 100 via Bluetooth in one embodiment, which uses less energy to conserve the battery life of the keypad. In various embodiments regarding the powering on and sleep mode for wireless keypad 122 (applicable to smartphone 120 also): (i) pressing any button of wireless keypad 122 will wake the keypad from sleep, (ii) upon waking, keypad 122 receives from PD machine 20 the current phase of treatment (fill, dwell or drain), and (iii) keypad 122 may go back to sleep if the current phase is not a drain phase. In various embodiments regarding display 132 for wireless keypad 122: (i) wireless keypad 122 will have a small liquid crystal display (“LCD”), light emitting diode display (“LED”) or other type of character display, (ii) during the powering of wireless keypad 122, display area 134 will briefly show a serial number of the keypad (e.g., for a few seconds), after which display area 134 will show the current negative drain pressure level (e.g., 1 to 10). Wireless keypad 122 may be configured to display the serial number when it is powered ON, while PD machine 20 provides a configuration screen to register keypad 122 via its serial number. In one embodiment, only one keypad 122 is allowed to pair with PD machine 20.

For any type of PDA, buttons 124a, 124b, 126a, 126b, 128, 130 and display screen 134 are provided in one embodiment as a mobile application downloadable and playable on smartphone 120 or notebook/wireless keypad 122. The mobile application allows smartphone 120 or notebook/wireless keypad 122 to transmit data to and receive data from PD machine 20. In one embodiment, the mobile application is provided with the tools to allow patient 12 to register or pair with PD machine 20, e.g., via WIFI (e.g., for smartphone 120 or notebook 122), Bluetooth (e.g., for keypad 122) or other wireless technology. Wireless communication between the mobile application on the patient's PDA and control unit 100 of PD machine 20 may take place in a variety of ways, for example, (i) via an infrastructure mode in which control unit 100 of PD machine 20 and the mobile application of the PDA are each connected to a home router and communication is through the home network, or (ii) via WIFI direct in which control unit 100 of PD machine 20 provides a WIFI access point to which the mobile application of the PDA connects in a peer-to-peer manner.

In one implementation, the mobile application is provided with a tab for registering with PD machine 20. After pressing the tab, the patient is allowed to enter into control unit 100 of PD machine 20 a unique number or to scan a QR code using the mobile application for registration. After registration, the version of the mobile application installed on smartphone 120 or notebook/wireless keypad 122 only communicates with the patient's PD machine 20. Memory 104 of control unit 100 may store a unique International Mobile Equipment Identity (“IMEI”) identifier to authenticate a drain pressure command from smartphone 120 or notebook/wireless keypad 122 before entering it into a machine control area of the control unit. Control unit 100 may also look for a secret code generated by the mobile application for authentication purposes. In an embodiment, only one mobile application may register with PD machine 20.

Once registered, the mobile application is connected automatically to PD machine 20 when both (i) smartphone 120 or notebook/wireless keypad 122 and (ii) PD machine 20 are powered. The mobile application may be configured to receive and display at display area 134 the maximum and minimum allowed drain pressure values configured in PD machine 20. The mobile application may be configured to have persistent memory that stores old maximum, minimum and default drain pressure values. The mobile application may be configured such that when opened, it displays at display area 134 the unique identification of its paired PD machine 20 along with the minimum and maximum negative pressure threshold values of the machine.

In one embodiment, Up button 124a and Down button 124b enable patient 12 to increase and decrease, respectively, the negative pressure or suction level provided by pump 26/72 (pneumatic) or pump 80 (electromechanical) between a minimum allowable negative pressure and a maximum allowable negative pressure. Display area 134 of smartphone 120 or notebook/wireless keypad 122 displays an incremented negative pressure level number, e.g., 1 to 10 in the illustrated embodiment, which is a relative scale, wherein 1 indicates the minimum allowable negative pressure, 10 indicates the maximum allowable negative pressure, and 2 to 9 range from the minimum to the maximum allowable negative pump pressures.

Up button 124a and Down button 124b increment the count one-by-one in the selected direction, e.g., from 4 to 5 or from 9 to 8. Pressing Min button 126a takes the level from wherever it is at directly to 1. If patient 12 for example begins to experience a lot of drain pain while PD machine 20 is pumping at a high negative pressure level, the patient may press Min button 126a and then the Send button 130, which causes pump 26/27 or pump 80 to modify its output such that the negative pressure is lowered from whatever level it is currently at to the minimum negative pressure level of 1. Pressing Max button 126b takes the level directly to 10. If patient 12 instead feels good and wants to speed-up the patient drain phase by raising the negative pressure level, the patient may press the Max button 126b and then the Send button 130, which causes pump 26/27 or pump 80 to modify its output such the negative pressure is raised from whatever level it is currently at to the maximum negative pressure level of 10. Negative pressure levels 1 to 10 are in one embodiment each within established safe patient pressure limits so that in no situation is it possible for PD machine 20 to supply too much negative pressure, potentially overpressurizing patient 12, or too little negative pressure, leading to an ineffective patient drain.

When a negative pressure value is displayed in display area 134 that the patient wishes to send to PD machine 20, the patient selects or presses the Send button 130, which causes the PDA to wirelessly deliver the new commanded negative pressure value to control unit 100 of PD machine 20. The patient may therefore increment negative pressure values as displayed at display area 134 without those values being sent to PD machine 20. Instead, the pressing of Send button 130 causes the negative pressure value currently displayed at display area 134 to be delivered to PD machine 20, which changes the pump pressure of used PD fluid being removed from the patient accordingly. Smartphone 120 or notebook/wireless keypad 122 may optionally display a popup confirm button after the patient presses Send button 130 to confirm that the patient wants a new negative pressure value to be delivered to PD machine 20.

The negative pressure values set as discussed above are communicated to PD cycler 20 wirelessly as discussed herein and continuously or according to some transmission frequency. Control unit 100 of PD machine automatically converts the mobile application values into corresponding negative pump pressures, e.g., via one or more lookup table stored in one or more memory 104. That is, the patient entered pressure level, e.g., 1 to 10, is converted to a pressure value, e.g., in pressure units such as psig, at control unit 100. In an alternative embodiment, the patient entered pressure level, e.g., 1 to 10, is converted to a pump actuation value, such as a variable orifice valve value (pneumatic) or a motor current value (electromechanical), which corresponds to the updated pressure value. In a further alternative embodiment, either of the above alternatives is performed at the PDA (smartphone 120 or notebook/wireless keypad 122).

The patient using system 10 may change the drain pressure values during a patient drain based on pain experienced, and do so gradually or via larger steps as discussed above. But the patient is in one embodiment not able to completely shut down the patient drain. If patient 12 still feels pain after PD machine 20 is run at the minimum negative pressure level, then patient is able to press Help button 128, which causes control unit 100 of PD machine 20 to alert a remote caretaker or clinic. The caretaker or clinic, which communicates with the control unit 100 of PD machine 20 over a network, provides additional steps or procedures to reduce drain pain.

Control unit 100 of PD machine 20 is configured to end a patient drain in a number of ways. In one way, control unit looks for a particular effluent drain volume, which is based on the PD fluid amount of the previous patient fill, an expected amount of ultrafiltration (“UF”) gained over the prior patient dwell, and any residual PD fluid volume built over the course of treatment. Here, control unit 100 is not monitoring patient drain time, so that if the patient lowers the negative drain pressure and slows the patient drain, control unit 100 allows the patient drain to run until the target effluent drain volume has been removed. In another embodiment, the patient drain is time-based so that if the patient lowers the negative drain pressure and slows the patient drain, control unit 100 extends the patient drain time accordingly so that the amount of effluent removed is the same as if the patient had not lowered the negative drain pressure and slowed the patient drain. In a third embodiment (which is combinable with the first two embodiments), control unit 100 looks for a characteristic increase or spike in drain pressure that indicates the patient is empty or almost empty, such that draining should stop. In any of the above embodiments, to reduce patient drain time, it is desirable to prompt the patient to increase drain pressure (if the patient feels good) in a manner discussed herein to speed the subsequent portion of the patient drain.

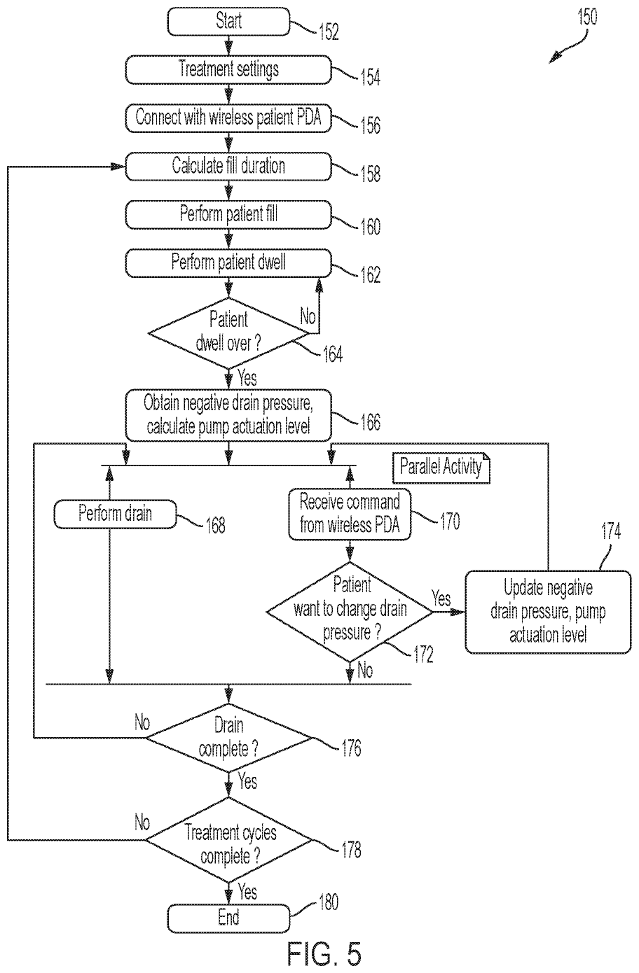

Referring now to FIG. 5, an embodiment for a PD treatment using the patient controlled negative drain pressure of the present disclosure is illustrated by method 150. Method 150 illustrates a PD treatment in which the patient begins treatment empty such that the first phase of the treatment is a patient fill. Many PD treatments begin instead with the patient full of used PD fluid, such that the first phase of the treatment is a patient drain. It should be appreciated that the patient drain control discussed herein and at the steps from diamond 164 to diamond 174 of method 150 may be performed equally as well for an initial patient drain that commences PD treatment.

At oval 152, method 150 begins. At block 154, treatment settings, such as fluid connections with PD fluid sources 40 and the loading of a disposable set (e.g., medical fluid handling device 70 of FIG. 1) to PD machine 20, are made. At block 156, an automatic data connection (assuming pairing or registration has already taken place) of the patient's PDA (smartphone 120 or notebook/wireless keypad 122) to control unit 100 of PD machine 20 is made.

At block 158, control unit 100 calculates a time needed for pump 26/27 or pump 80 to perform a patient fill. At block 160, control unit 100 causes pump 26/27 or pump 80 to be actuated and sequences any needed valves to perform a patient fill. At block 160, control unit 100 causes pump 26/27 or pump 80 to stop and sequences any needed valves to perform a patient dwell. At diamond 164, control unit 100 determines of the patient dwell has been completed. If not, then method 150 returns to block 162. If the patient dwell has been completed as determined at diamond 164, control unit 100 proceeds to performing a patient drain according to system 10 of the present disclosure.

At block 166, control unit 100 causes a patient drain to begin by obtaining an initial negative drain pressure and calculating or determining a corresponding level for actuating pump 26/27 or pump 80. Control unit 100 for the patient drain splits into parallel paths. In one path at block 168 no patient command is received from the patient's PDA, such that control unit 100 causes the patient drain to proceed with pump 26/27 or pump 80 actuated at the initially obtained level.

In a second path at block 170, control unit 100 receives a wireless command from the patient's PDA (smartphone 120 or notebook/wireless keypad 122). At diamond 172, control unit determines whether the wireless command is a command to change the negative patient drain pressure (e.g., versus a Help button command). If not, then control unit 100 performs whatever task is associated with the wireless command and the patient drain proceeds according block 168 of the first path. If the command does involve a change in the negative patient drain pressure as determined at diamond 172, then method proceeds to block 174.

At block 174, control unit 100 updates the patient drain by updating the negative drain pressure according to the newly received command from the patient (up or down) and calculates or determines a new corresponding level for actuating pump 26/27 or pump 80. Method 150 then returns to where control unit 100 splits into parallel paths, one to block 168 and the other to block 170.

At diamond 176, control unit 100 determines if the present drain has been completed (according to any one or more way discussed above). If not, then method 150 returns to where control unit 100 splits into parallel paths, one to block 168 and the other to block 170. If the present drain has been completed (according to any one or more way discussed above), as determined at diamond 176, then method 150 proceeds to diamond 178.

At diamond 178, control unit 100 determines if all treatment cycles for the present treatment have been completed. If not, then method 150 returns to block 158 to begin the next patient fill. If all treatment cycles for the present treatment have been completed, as determined at diamond 178, then method 150 ends at oval 180.

It should be understood that various changes and modifications to the presently preferred embodiments described herein will be apparent to those skilled in the art. It is therefore intended that such changes and modifications be covered by the appended claims. For example, while system 10 and its associated methodology have been discussed as providing the patient with negative drain pressure control, system 10 may additionally provide the patient with positive fill pressure control.

1: A peritoneal dialysis (“PD”) system comprising:
a PD fluid pump;
a patient line for receiving used PD fluid pumped by the PD fluid pump during a patient drain;
a control unit configured to cause the PD fluid pump to pump, according to a first commanded pressure, the used PD fluid through the patient line during the patient drain; and
a personal digital assistant (“PDA”) in wireless communication with the control unit, the PDA configured to enable a patient while undergoing the patient drain to send a command to the control unit, the command instructing the control unit to cause the PD fluid pump to pump the used PD fluid according to a second commanded pressure during the patient drain,
wherein the PDA provides a set of relative pressure values from which the patient may choose for sending the command, a selected pressure value being limited to between a minimum allowable pressure value and a maximum allowable pressure value, the maximum allowable pressure value being greater than a default pressure value.

2: The PD system of claim 1, wherein the second commanded pressure is less than the first commanded pressure, the second commanded pressure aiding in the reduction of pain experienced by the patient during the patient drain.

3: The PD system of claim 1, wherein the second commanded pressure is greater than the first commanded pressure, the second commanded pressure aiding in reducing a duration of the patient drain.

4: The PD system of claim 1, wherein the control unit is configured to convert a selected relative pressure value to the second commanded pressure.

5: The PD system of claim 1, wherein the control unit is configured to convert a selected relative pressure value to a pump actuation value that corresponds to the second commanded pressure.

6: The PD system of claim 1, wherein the PDA is configured to convert a selected relative pressure value to the second commanded pressure.

7: The PD system of claim 1, wherein the PDA is configured to convert a selected relative pressure value to a pump actuation value that corresponds to the second commanded pressure.

8: The PD system of claim 1, wherein the PDA provides at least one of an incremental up selector or an incremental down selector for incrementing through the set of relative pressure values.

9: The PD system of claim 8, wherein the PDA further provides a display for displaying an incremented relative pressure value.

10: The PD system of claim 8, wherein the PDA further provides a selector for causing an incremented relative pressure value desired by the patient, or a conversion of the incremented relative pressure value, to be delivered to the control unit.

11: The PD system of claim 8, wherein the PDA further provides a selector for moving through the set of relative pressure values to the minimum allowable pressure value.

12: The PD system of claim 8, wherein the PDA further provides a selector for moving through the set of relative pressure values to the maximum allowable pressure value.

13: The PD system of claim 1, wherein the PD fluid pump is pneumatically operated or electromechanically operated.

14: The PD system of claim 1, wherein the PDA is configured to be registered with the control unit such that after registration, the PDA and the control unit communicate wirelessly automatically upon being powered.

15: The PD system of claim 14, wherein the PDA is configured to be registered with the control unit via a serial number or QR code from the PDA being entered into the control unit.

16: The PD system of claim 1, wherein the PDA runs a mobile application configured to enable the patient while undergoing the patient drain to send the command to the control unit.

17: The PD system of claim 1, wherein the command is a first command, wherein the control unit is further configured to communicate with a patient clinic over a network, and wherein the PDA further provides a selector configured such that when selected a second command is generated requesting that the control unit contact the patient clinic.

18: The PD system of claim 1, wherein the PD fluid pump and the control unit are part of a PD machine, and wherein the PDA is remote from the PD machine.

19: A peritoneal dialysis (“PD”) system comprising:
a PD fluid pump;
a patient line for receiving PD fluid pumped by the PD fluid pump during a PD treatment procedure;
a control unit configured to cause the PD fluid pump to pump, according to a first commanded pressure, the PD fluid through the patient line during the PD treatment procedure; and
a personal digital assistant (“PDA”) in wireless communication with the control unit, the PDA configured to enable a patient while undergoing the PD treatment procedure to send a command to the control unit, the command instructing the control unit to cause the PD fluid pump to pump PD fluid according to a second commanded pressure during the PD treatment procedure,
wherein the PDA provides a set of relative pressure values from which the patient may choose for sending the command, a selected pressure value being limited to between a minimum allowable pressure value and a maximum allowable pressure value, the maximum allowable pressure value being greater than a default pressure value.

20: The PD system of claim 19, wherein the PD fluid is used PD fluid and the PD treatment procedure is a patient drain.

21: A peritoneal dialysis (“PD”) machine comprising:
a PD fluid pump;
a patient line for receiving used PD fluid pumped by the PD fluid pump during a patient drain;
a control unit configured to cause the PD fluid pump to pump, according to a first commanded pressure, the used PD fluid through the patient line during the patient drain; and
a user interface in communication with the control unit, the user interface configured to enable a patient while undergoing the patient drain to send a command to the control unit, the user interface command instructing the control unit to cause the PD fluid pump to pump the used PD fluid according to a second commanded pressure during the patient drain,
wherein the user interface provides a set of relative pressure values from which the patient may choose for sending the command, a selected pressure value being limited to between a minimum allowable pressure value and a maximum allowable pressure value, the maximum allowable pressure value being greater than a default pressure value.

22: A PD system using the PD machine of claim 21, wherein the PD system includes a personal digital assistant (“PDA”) in wireless communication with the control unit, the PDA also configured to enable the patient while undergoing the patient drain to send a PDA command to the control unit, the PDA command instructing the control unit to cause the PD fluid pump to pump the used PD fluid according to a second commanded pressure during the patient drain.
